(19) United States
(12) Patent Application Publication
(10) Pub. No.: US 2022/0331482 A1
(43) Pub. Date: Oct. 20, 2022

(54) Hydrogel Composition For Tissue Regeneration, And Support Prepared Using Same

(71) Applicant: REGEN BIOCHARM CO., LTD., Seongnam-si, Gyeonggi-do (KR)

(72) Inventors: Gyung Gyun KIM, Seoul (KR); Myung CHOI, Seoul (KR); Sang Jin KIM, Busan (KR)

(21) Appl. No.: 17/763,136

(22) PCT Filed: Sep. 1, 2020
(86) PCT No.: PCT/KR2020/011731

§ 371 Date: Mar. 23, 2022

(30) Foreign Application Priority Data
Sep. 26, 2019 (KR) 10-2019-0118641

(51) Int. Cl. A61L 27/20; A61L 27/52; A61L 27/38; A61K 31/728; A61L 27/24

(52) U.S. Cl. CPC A61L 27/20; A61L 27/52; A61L 27/38; A61K 31/728; A61L 27/24; A61L 27/3813; A61L 2430/00

(57) ABSTRACT

The present invention relates to a hydrogel composition for tissue regeneration, and a support prepared using same. More specifically, the present invention comprises anionic polysaccharides, aminated hyaluronic acid, and collagen. The hydrogel composition comprising the ingredients, and a support prepared using same can quickly form a three-dimensional structure through spontaneous cross-linking without the addition of a common cross-linking agent in which an epoxide group or an amine group is at a single end or both ends thereof, thereby providing a structure capable of soft tissue transplants.

### TECHNICAL FIELD

The present invention relates to a hydrogel composition for tissue regeneration and a support prepared using the same, and more particularly to a hydrogel composition for tissue regeneration, which provides a structure capable of soft tissue transplantation because it is possible to rapidly form a three-dimensional structure through spontaneous cross-linking without introduction of a cross-linking agent having an epoxide group or an amine group at one or both ends thereof, and a support prepared using the same.

### BACKGROUND ART

At present, a main goal of tissue engineering is to replace damaged tissues and organs by forming functional human tissues and organs composed of various cells. Bioprinting ink is capable of helping quickly realize the goal of tissue engineering, and bioprinting is performed to form tissues and organs having a desired three-dimensional structure using cells and biomaterials based on automated bioprinter technology. Automated computer bioprinting technology has been developed through an inkjet-based process, a laser-based process, and an extrusion-based process. The basic process of bioprinting includes modeling an image obtained from a computer-aided design model and creating a three-dimensional structure using bioink that contains cells and materials. Computer-aided design models may be created using nuclear magnetic resonance images and 3D medical images through computerized phase scanning. Here, biomaterials including living cells are used as basic materials for the bioprinting process.

In order for 3D printed biomimetic constructs including cells to function in structural and biological aspects, bioink has to possess printability, cell compatibility, biodegradability, desired gelation properties/mechanical properties, and capability to regulate cell growth and differentiation.

Specifically, the bioprinted structure has to maintain a desired shape during regeneration and to decompose at an appropriate rate during regeneration in vivo. Examples of currently useful bioink include scaffold-based hydrogels, microcarriers, extracellular matrixes from which cells have been removed, and the like, and cell aggregates having no scaffolds may also be used as bioink.

A hydrogel, which is a representative commercialized bioink, has superior biocompatibility and a structure similar to human tissue, and facilitates encapsulation of cells and physiologically active materials. Typical examples of a hydrogel bioink product include collagen, gelatin, alginate, hyaluronic acid, poly(ethylene glycol) diacrylate (PEGDA), collagen methacryloyl (CollagenMA), gelatin methacryloyl (GelMA), and the like.

Extrusion-based printing is the most commonly used method in 3D bioprinting, and most commercialized 3D bioprinters use an extrusion-based printing process. Extrusion-based printing is performed in a manner in which bioink in a syringe is extruded in a fine thread form using pneumatic or mechanical force to produce a 3D cell construct. Extrusion-based printing is capable of using bioink having a wide range of viscosities (30-6×10⁶ mPa·s) and high concentrations of cells and cell spheroids. However, extrusion-based printing has a low resolution (200-1000 μm), and is likely to apply shear stress to cells during the extrusion process, which may affect the viability of cells. Therefore, bioink used for extrusion-based printing must have shear thinning properties, maintain the printed form well after printing, and protect cells from shear stress.

Inkjet-based printing is based on the principle by which small droplets (10-50 μm) are piezoelectrically or thermally generated from bioink including cells and sprayed through a nozzle. During the inkjet-based printing process, the cells in the bioink are exposed to high heat in a short period (2 μs), but cell viability is not greatly affected. In inkjet-based printing, bioink has to have low viscosity (less than 10 mPas) and a low cell concentration (less than 10⁶ cells/ml), which is undesirable.

Laser-based printing does not require a nozzle, so there is no clogging caused by a nozzle, and the cells contained in bioink are not exposed to shear stress because bioink does not pass through a nozzle. Laser-based printing is based on the principle of generating and propelling droplets by projecting a pulsed laser beam onto a donor ribbon composed of an absorption layer of gold or titanium and a bioink layer. Laser-based printing is capable of using bioink having a viscosity of 1-300 mPas and a cell concentration of 10⁸ cells/ml, and has a resolution of 10-100 μm. A high-energy laser is able to temporarily provide heat to the bioink, so bioink having low thermal conductivity is capable of improving the viability of cells in the ink.

Currently useful printing processes are alike with regard to the importance of bioink using biocompatible materials.

Such a three-dimensional support is manufactured using a material for precisely forming a tissue-like organ and a transplantable construct. This technology makes it possible to create small and large tissue constructs that mimic real human tissues almost perfectly.

However, the support for transporting these cells, that is, bioink, has many limitations on use thereof. In order to broaden the range of use, which is a limitation of these biomaterials, there is need for a hydrogel complex useful in processes involving bioprinting and injection.

### Technical Problem

It is an object of the present invention to provide a hydrogel composition for tissue regeneration, which provides a structure capable of soft tissue transplantation because it is possible to rapidly form a three-dimensional structure through spontaneous cross-linking without introduction of a cross-linking agent having an epoxide group or an amine group at one or both ends thereof, and a support prepared using the same.

### Technical Solution

In order to accomplish the above object, the present invention provides a hydrogel composition for tissue regeneration including an anionic polysaccharide, aminated hyaluronic acid, and collagen.

In a preferred embodiment of the present invention, the hydrogel composition for tissue regeneration includes 1 to 10 wt % of an anionic polysaccharide, 0.1 to 2 wt % of aminated hyaluronic acid, and 0.1 to 3 wt % of collagen.

In a more preferred embodiment of the present invention, the anionic polysaccharide has a weight average molecular weight of 100 to 500 kDa and is composed of 50 to wt % of β-D-mannuronic acid and 30 to 50 wt % of α-L-guluronic acid.

In a more preferred embodiment of the present invention, the aminated hyaluronic acid has a weight average molecular weight of 100 to 500 kDa.

In a more preferred embodiment of the present invention, the collagen has a weight average molecular weight of 100 to 500 kDa and is composed of atelocollagen.

In a more preferred embodiment of the present invention, the hydrogel composition for tissue regeneration further includes 150 to 400 parts by weight of a divalent cation and 0.1 to 2 parts by weight of methanol based on 100 parts by weight of the anionic polysaccharide contained in the hydrogel composition for tissue regeneration.

In a more preferred embodiment of the present invention, the divalent cation has a mass concentration of 0.5 to 2% and is composed of an alkaline earth metal or a compound thereof.

In an even more preferred embodiment of the present invention, the methanol has a mass concentration of 40 to 60%.

In an even more preferred embodiment of the present invention, the hydrogel composition for tissue regeneration further includes 0.1 to 1 parts by weight of an additive based on 100 parts by weight of the hydrogel composition for tissue regeneration, the additive including at least one selected from the group consisting of a cell, cell growth factor, buffer, preservative, isotonicity regulator, salt, antioxidant, osmotic pressure regulator, emulsifier, wetting agent, sweetener, flavoring agent, and anesthetic agent.

In an even more preferred embodiment of the present invention, the cell includes at least one selected from the group consisting of a stem cell, sensory cell, brain cell, germ cell, epithelial cell, and immune cell.

In addition, the present invention provides a hydrogel support for tissue regeneration, prepared using the hydrogel composition for tissue regeneration.

### Advantageous Effects

According to the present invention, a hydrogel composition for tissue regeneration and a support prepared using the same are capable of quickly forming a three-dimensional structure through spontaneous cross-linking without introduction of a cross-linking agent having an epoxide group or an amine group at one or both ends thereof, and are thus very effective at providing a structure capable of soft tissue transplantation.

### DESCRIPTION OF DRAWINGS

FIG. 1 is a photograph showing a hydrogel support prepared in Preparation Example 5 of the present invention;

FIG. 2 is a schematic view showing a process of preparing a hydrogel support in Preparation Example 5 of the present invention;

FIG. 3 is a graph showing the results of measurement of compressive strength depending on the concentration of a divalent cation;

FIG. 4 is a photograph showing a hydrogel composition prepared in Preparation Example 3 of the present invention;

FIG. 5 is an optical microscope image showing the hydrogel composition freeze-dried after preparation in Preparation Example 3; and

FIG. 6 is an optical microscope image showing the hydrogel support freeze-dried after preparation in Preparation Example 5 of the present invention.

### MODE FOR INVENTION

Hereinafter, preferred embodiments of the present invention and the physical properties of individual components will be specifically described, which is intended to provide a sufficiently detailed explanation that those skilled in the art to which the present invention pertains could easily carry out the invention, and which is not to be construed as limiting the technical spirit and scope of the present invention.

According to the present invention, a hydrogel composition for tissue regeneration includes an anionic polysaccharide, aminated hyaluronic acid, and collagen, and preferably includes 1 to 10 wt % of an anionic polysaccharide, 0.1 to 2 wt % of aminated hyaluronic acid, and 0.1 to 3 wt % of collagen.

The hydrogel composed of anionic polysaccharide, aminated hyaluronic acid, and collagen as described above is able to exhibit mechanical properties of rapidly realizing cross-linking and maintaining the printed structure, and also to be injected into the skin soft tissue, thereby overcoming conventional limitations on biomaterials.

Moreover, the hydrogel composition composed of the above components may be added with a tissue-derived extracellular matrix component or with a specific cell differentiation regulator to induce differentiation into specific tissues.

As used herein, the term “hydrogel” refers to a water-swellable polymer that is usable as a synthetic material for biotissue as well as drug delivery. Such a water-swellable polymer is a polymer that absorbs water but does not dissolve in water, namely a hydrophilic polymer having sufficient expansion in water, and may be blended with cells and growth factors to provide a more biocompatible support.

The anionic polysaccharide is contained in an amount of 1 to 10 wt %, has a weight average molecular weight of 100 to 500 kDa, and is composed of 50 to 70 wt % of β-D-mannuronic acid and 30 to 50 wt % of α-L-guluronic acid. The amount of the anionic polysaccharide accounts for 1 to 10 wt %, preferably 4 to 10 wt %, of the total amount of the hydrogel composition for tissue regeneration according to the present invention.

If the amount of the anionic polysaccharide is less than 1 wt % or exceeds 10 wt %, an egg box cannot be formed when a divalent cation is added, or a water-insoluble hydrogel cannot be formed even when an egg box is formed locally.

The aminated hyaluronic acid is contained in an amount of 0.1 to 2 wt % and has a weight average molecular weight of 100 to 500 kDa. It is preferable to use aminated hyaluronic acid in which at least a portion of the hydroxyl group hydrogen atoms of hyaluronic acid is substituted with a group having a quaternary ammonium cation group.

As described above, the hyaluronic acid having a quaternary ammonium cation group is contained in an amount of 0.1 to 2 wt %, preferably 0.5 to 1 wt %, in the first hydrogel. If the amount of aminated hyaluronic acid exceeds 2 wt %, a polyion complex may be formed by electrostatic interaction between the anionic polysaccharide of the hydrogel composition and the aminated hyaluronic acid due to coexistence, so a locally non-uniform water-insoluble gel may precipitate. On the other hand, if the amount of aminated hyaluronic acid is less than 0.1 wt %, a 13 sheet with collagen may not be properly formed in the solvent.

The collagen is contained in an amount of 0.1 to 3 wt %, has a weight average molecular weight of 100 to 500 kDa, and is composed of atelocollagen. Here, it may include any one or more selected from among type-1 atelocollagen, type-2 atelocollagen, and type-3 atelocollagen, and it is preferable to use collagen in which the end of the functional group thereof is substituted with succinic acid or a sulfide bond.

The collagen is contained in an amount of 0.1 to 3 wt %, preferably 0.5 to 1.5 wt % in the hydrogel composition for tissue regeneration. If the amount of collagen exceeds wt %, conversion into the 13 sheet may occur drastically due to the solvent and partial non-uniformity may result, making it impossible to prepare a uniform hydrogel composition and support. On the other hand, if the amount of collagen is less than 0.1 wt %, a uniform shape may be obtained in a hydrogel composition state, but non-uniform fibrosis may occur during preparation of the support.

Also, the hydrogel composition for tissue regeneration according to the present invention may further include 150 to 400 parts by weight of a divalent cation and 0.1 to 2 parts by weight of methanol based on 100 parts by weight of the anionic polysaccharide contained in the hydrogel composition for tissue regeneration. When the cation is further contained in this way, the hydrogel composition for tissue regeneration according to the present invention is insolubilized in water.

Here, the divalent cation has a mass concentration of 0.5 to 2%, and preferably includes an alkaline earth metal or a compound thereof. More preferably, the alkaline earth metal includes beryllium, magnesium, calcium, strontium, barium, and radium.

In addition, the methanol serves to change the structure of the hydrogel composition in the process of insolubilizing the hydrogel composition for tissue regeneration in water as described above, and preferably has a mass concentration of 40 to 60%.

The process of insolubilizing the hydrogel composition in water by mixing the divalent cation and methanol is not particularly limited, but it is preferable to select and use a preparation method suitable for realizing high product uniformity and mass production. More preferably, it is performed in a manner in which the hydrogel composition is prepared, a divalent cation is sprayed thereto, methanol is added dropwise thereto, and then a negative pressure of −0.05 MPa is applied thereto at a temperature of 0 to 60° C. to remove the methanol.

Here, the conditions of temperature and negative pressure do not limit the scope of the present invention, and may be changed in various ways, and the methanol removal process is not limited to the temperature and negative pressure conditions, and a removal process using a dialysis membrane is also possible.

Moreover, the hydrogel composition for tissue regeneration according to the present invention may further include 0.1 to 1 parts by weight of an additive based on 100 parts by weight of the hydrogel composition for tissue regeneration, and the additive includes at least one selected from the group consisting of a cell, cell growth factor, buffer, preservative, isotonicity regulator, salt, antioxidant, osmotic pressure regulator, emulsifier, wetting agent, sweetener, flavoring agent, and anesthetic agent.

Here, the cell may include at least one selected from the group consisting of a stem cell, sensory cell, brain cell, germ cell, epithelial cell, and immune cell.

Also, the anesthetic agent may be, for example, a local anesthetic agent such as an aminoamide local anesthetic agent or an aminoester local anesthetic agent. Examples of the local anesthetic agent may include, but are not limited to, lidocaine, ambucaine, amolanone, amylocaine, benoxinate, benzocaine, betoxycaine, biphenamine, bupivacaine, butacaine, butamben, butanilicaine, butethamine, butoxycaine, carticaine, chloroprocaine, cocaethylene, cyclomethycaine, dibucaine, dimethisoquin, dimethocaine, diperodon, dicyclomine, ecgonidine, ecgonine, ethyl chloride, etidocaine, β-eucaine, euprocin, fenalcomine, formocaine, hexylcaine, hydroxytetracaine, isobutyl p-aminobenzoate, leucinocaine mesylate, levoxadrol, lidocaine, mepivacaine, meprylcaine, metabutoxycaine, methyl chloride, myrtecaine, naepaine, octacaine, orthocaine, oxethazaine, parethoxycaine, phenacaine, phenol, piperocaine, piridocaine, polidocanol, pramoxine, prilocaine, procaine, propanocaine, proparacaine, propipocaine, propoxycaine, pseudococaine, pyrrocaine, ropivacaine, salicyl alcohol, tetracaine, tolycaine, trimecaine, zolamine, combinations thereof, and salts thereof. Examples of the aminoester local anesthetic agent may include, but are not limited to, procaine, chloroprocaine, cocaine, cyclomethycaine, dimethocaine (larocaine), propoxycaine, procaine (Novocain), proparacaine, and tetracaine (amethocaine). Non-limiting examples of the aminoamide local anesthetic agent include articaine, bupivacaine, cinchocaine (dibucaine), etidocaine, levobupivacaine, lidocaine (lignocaine), mepivacaine, piperocaine, prilocaine, ropivacaine, trimecaine, and combinations thereof.

When the additive described above is contained, the hydrogel composition according to the present invention may be used for improving the condition of the skin, filling wrinkles, or shaping the face or body, and is also useful as a dermal filler.

Hereinafter, a method of preparing a hydrogel composition for tissue regeneration according to the present invention, a method of preparing a support using a hydrogel composition prepared through the above method, and individual physical properties will be described with reference to the following examples.

### <Preparation Example 1> Preparation of Hydrogel Composition for Injection

A hydrogel composition for injection was prepared in a manner in which 7 wt % of an anionic polysaccharide having a weight average molecular weight of 100 kDa to 500 kDa (obtained by mixing β-D-mannuronic acid and α-L-guluronic acid at a ratio of 50:50 at room temperature) was mixed with 0.7 wt % of aminated hyaluronic acid having a weight average molecular weight of 100 kDa to 500 kDa to afford a mixture, and the pH of the mixture was maintained at 6.0, followed by adding 1 wt % of type-1 atelocollagen having a weight average molecular weight of 100 kDa to 500 kDa thereto.

### <Preparation Example 2> Preparation of Hydrogel Composition for Injection Containing Additive

A hydrogel composition for injection containing an additive was prepared by mixing 100 parts by weight of the hydrogel composition for injection prepared in Preparation Example 1 with 0.5 parts by weight of an additive (including a buffer, an osmotic pressure regulator, an emulsifier, and lidocaine, which are mixed together).

### <Preparation Example 3> Preparation of Water-Insoluble Hydrogel Composition for Injection

A water-insoluble hydrogel composition for injection was prepared in a manner in which the hydrogel composition prepared in Preparation Example 1 was mixed with calcium chloride having a mass concentration of 1% at room temperature, allowed to react with stirring at a rate of 100 rpm, added with methanol having a mass concentration of 60%, and then stirred at room temperature at a rate of 500 rpm, followed by removal of residual methanol using a dialysis membrane or a rotary vacuum evaporator.

### <Preparation Example 4> Preparation of Water-Insoluble Hydrogel Composition for Injection Containing Additive

A water-insoluble hydrogel composition for injection was prepared in a manner in which the hydrogel composition prepared in Preparation Example 2 was mixed with calcium chloride having a mass concentration of 1% at room temperature, allowed to react with stirring at a rate of 100 rpm, added with methanol having a mass concentration of 60%, and then stirred at room temperature at a rate of 500 rpm, followed by removal of residual methanol using a dialysis membrane or a rotary vacuum evaporator.

### <Preparation Example 5> Preparation of Hydrogel Support for 3D Printer

A hydrogel support for a 3D printer was prepared in a manner in which 7 wt % of a first solution (obtained by mixing β-D-mannuronic acid and α-L-guluronic acid having a weight average molecular weight of 100 kDa to 500 kDa at a rate of 50:50 at room temperature) was mixed with calcium chloride having a mass concentration of 1% at room temperature, allowed to react with stirring at a rate of 100 rpm, and dried to afford a support, and the support thus obtained was immersed in a second solution (obtained by mixing aminated hyaluronic acid having a weight average molecular weight of 100 kDa to 500 kDa, the pH of which was maintained at 6.0, with 1 wt % of type-1 atelocollagen having a weight average molecular weight of 100 kDa to 500 kDa) so that the second solution penetrated the support, after which the support having the second solution therein was immersed in methanol having a mass concentration of 60% and dried in a vacuum.

### <Preparation Example 6> Preparation of Hydrogel Support for 3D Printer Containing Additive

A hydrogel support for a 3D printer containing an additive was prepared in the same manner as Preparation Example 5, with the exception that the first solution was prepared by adding a mixture, obtained by mixing β-D-mannuronic acid and α-L-guluronic acid having a weight average molecular weight of 100 kDa to 500 kDa at a ratio of 50:50 at room temperature, with 0.5 parts by weight of an additive (including a buffer, an osmotic pressure regulator, an emulsifier, and lidocaine, which are mixed together).

### <Example 1> Preparation Depending on Concentration of Divalent Cation

Changes in a solid state depending on the concentration of the divalent cation were measured, and the results thereof are shown in Table 1 below.

TABLE 1

| | | A | | | | | |
|---|---|---|---|---|---|---|---|
| Classification | | 0.0% | 0.5% | 1.0% | | 1.5% | 2.0% |
| B | 4% | | | 1 | sec | 5 secs | |
| | 6% | | 1 sec | 3 | secs | 5+ secs | |
| | 8% | | 3 secs | 5+ | secs | | |
| | 10% | | 5 secs | | | | |

A: concentration of CaC1₂ (w/v)
B: concentration of mixture of β-D-mannuronic acid and α-L-guluronic acid (w/v)
Table color: White is a liquid state, and as the color darkens, the liquid changes to a solid state. (sol −> sold)

Table 1 shows the state change depending on the concentration of the divalent cation and the concentration of the mixture of β-D-mannuronic acid and α-L-guluronic acid. When the concentration of the mixture of β-D-mannuronic acid and α-L-guluronic acid was 4 wt % in the first hydrogel and the mass concentration of the calcium ion was 1.0 to 1.5%, the state shown in FIG. 4 resulted.

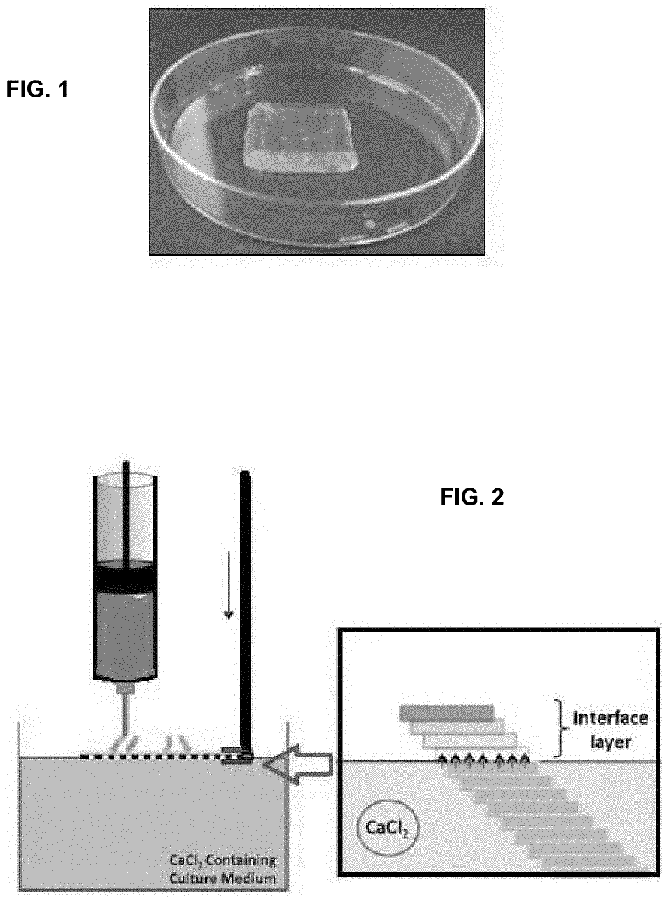

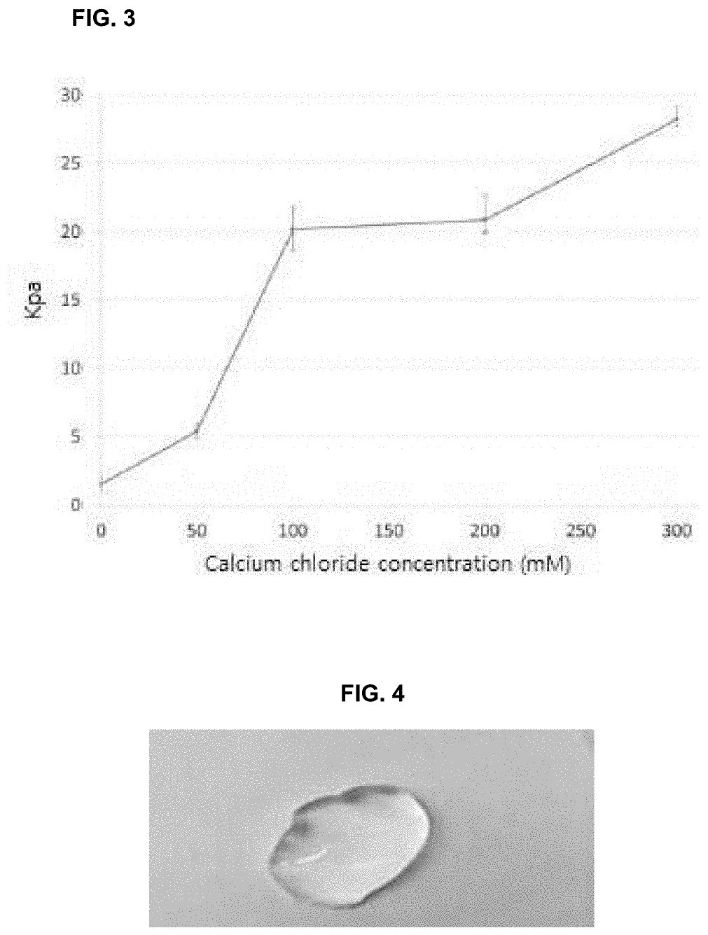

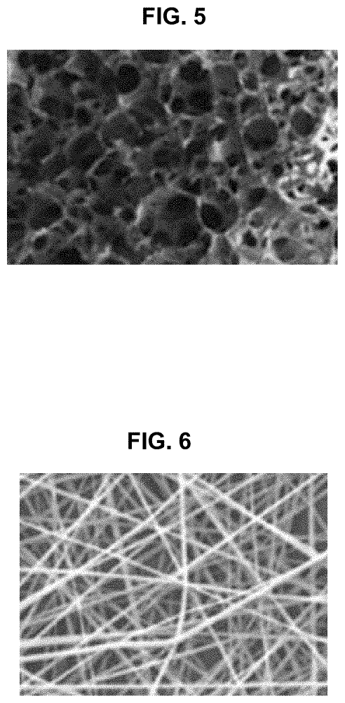

In addition, when the concentration of the mixture of β-D-mannuronic acid and α-L-guluronic acid was 6 wt % in the hydrogel composition and the mass concentration of the calcium ion was 0.5 to 1.0%, the state shown in FIG. 4 resulted. When the mass concentration of the calcium ion was 1.5%, a hard gel was formed, as shown in FIG. 1.

In addition, when the concentration of the mixture of β-D-mannuronic acid and α-L-guluronic acid was 8 wt % in the hydrogel composition and the mass concentration of the calcium ion was 0.5%, the state shown in FIG. 4 resulted. When the concentration of the calcium ion was 1.0%, a hard gel was formed, as shown in FIG. 1.

In addition, when the concentration of the mixture of β-D-mannuronic acid and α-L-guluronic acid was 10 wt % in the hydrogel composition and the mass concentration of the calcium ion was 0.5%, the state shown in FIG. 4 resulted.

As is apparent from Table 1, application of the hydrogel composition to a support for injection or a support for a 3D printer is determined depending on the concentration of the mixture of β-D-mannuronic acid and α-L-guluronic acid and the concentration of the divalent cation. Here, the calcium ion was added at a ratio of 3:7 relative to the mixture of β-D-mannuronic acid and α-L-guluronic acid.

### <Example 2> Change in Morphology Depending on Concentration of Divalent Cation

When the concentration of the mixture of β-D-mannuronic acid and α-L-guluronic acid was each of 4, 6, 8, and 10 wt % in the hydrogel composition in Example 1, changes in morphology depending on the concentration of the divalent cation were measured.

TABLE 2

| | B | | | | | |
|---|---|---|---|---|---|---|
| | 50 | 100 | 150 | 200 | 250 | 300 |
| A | mM | mM | mM | mM | mM | mM |
| 4% | 5 kPa | 5 kPa | 6 kPa | 21 kPa | 21 kPa | 26 kPa |
| 6% | 5 kPa | 20 kPa | 21 kPa | 21 kPa | 25 kPa | 29 kPa |
| 8% | | 28 kPa | 34 kPa | — | — | — |
| 10% | — | 35 kPa | — | — | — | — |

A: concentration of mixture of β-D-mannuronic acid and α-L-guluronic acid
B: concentration mM of calcium ion
—: Impossible to measure due to non-uniform gel state

As is apparent from Table 2, the tensile strength of the gel increased in most cases depending on the concentration of the mixture of β-D-mannuronic acid and α-L-guluronic acid and the concentration of the divalent cation. However, when both the concentration of the mixture and the concentration of the divalent cation were increased, non-uniform gelation progressed due to drastic egg box formation between the internal carboxyl groups of β-D-mannuronic acid and α-L-guluronic acid and the divalent cation.

### <Example 3> Change in Morphology of Mixture of β-D-Mannuronic Acid, α-L-Guluronic Acid, Aminated Hyaluronic Acid, and Collagen

TABLE 3

| | B | | | | | |
|---|---|---|---|---|---|---|
| | 50 | 100 | 150 | 200 | 250 | 300 |
| A | mM | mM | mM | mM | mM | mM |
| 4% | 6 kPa | 6 kPa | 7 kPa | 22 kPa | 23 kPa | 26 kPa |
| (+0.5% + | | | | | | |
| 1.0%) | | | | | | |
| 6% | 6 kPa | 20 kPa | 21 kPa | 22 kPa | 25 kPa | 30 kPa |
| (+0.5% + | | | | | | |
| 1.0%) | | | | | | |
| 8% | | 27 kPa | 35 kPa | — | — | — |
| (+0.5% + | | | | | | |
| 1.0%) | | | | | | |
| 10% | — | 34 kPa | — | — | — | — |
| (+0.5% + | | | | | | |
| 1.0%) | | | | | | |

A: concentration of mixture of β-D-mannuronic acid and α-L-guluronic acid uniformly added with 0.5% aminated hyaluronic acid and 1.0% collagen
B: concentration mM of calcium ion
—: Impossible to measure due to non-uniform gel state

As is apparent from Table 3, the tensile strength of the gel increased in most cases depending on the concentration of the mixture of β-D-mannuronic acid and α-L-guluronic acid and the concentration of the divalent cation, like Table 2. However, when both the concentration of the mixture and the concentration of the divalent cation were increased, non-uniform gelation progressed due to drastic egg box formation between the internal carboxyl groups of β-D-mannuronic acid and α-L-guluronic acid and the divalent cation, indicating that egg box formation is not affected by the divalent cation concentration.

### <Test Example 1> Cytotoxicity Test of Hydrogel of Preparation Example 3

The hydrogel composition prepared in Preparation Example 3 was dissolved in an amount of 10 wt % in PBS, and this mixture was added with 1×10⁶/100 μl of fibroblasts. The solution containing cells was cultured for 3 days in a DEME medium (10% FBS and 1% antibiotic), after which a cytotoxicity test was performed thereon using a Live and Dead kit.

Consequently, the average cell viability after 3 days was observed to be 96% or more.

### <Test Example 2> Cytotoxicity Test of Hydrogel Support of Preparation Example 5

The hydrogel support prepared in Preparation Example 5 was dissolved in an amount of 10 wt % in PBS, and this mixture was added with 1×10⁶/100 μl of fibroblasts. The solution containing cells was cultured for 3 days in a DEME medium (10% FBS and 1% antibiotic), after which a cytotoxicity test was performed thereon using a Live and Dead kit.

Consequently, the average cell viability after 3 days was observed to be 98% or more.

1. A hydrogel composition for tissue regeneration comprising 1 to 10 wt % of an anionic polysaccharide, 0.1 to 2 wt % of aminated hyaluronic acid, and 0.1 to 3 wt % of collagen, and further comprising 150 to 400 parts by weight of a divalent cation and 0.1 to 2 parts by weight of methanol based on 100 parts by weight of the anionic polysaccharide.

2. The hydrogel composition according to claim 1, wherein the anionic polysaccharide has a weight average molecular weight of 100 to 500 kDa and comprises 50 to 70 wt % of (β-D-mannuronic acid and 30 to 50 wt % of α-L-guluronic acid.

3. The hydrogel composition according to claim 1, wherein the aminated hyaluronic acid has a weight average molecular weight of 100 to 500 kDa.

4. The hydrogel composition according to claim 1, wherein the collagen has a weight average molecular weight of 100 to 500 kDa and comprises atelocollagen.

5. The hydrogel composition according to claim 1, wherein the divalent cation has a mass concentration of 0.5 to 2% and comprises an alkaline earth metal or a compound thereof.

6. The hydrogel composition according to claim 1, wherein the methanol has a mass concentration of 40 to 60%.

7. The hydrogel composition according to claim 1, further comprising 0.1 to 1 parts by weight of an additive based on 100 parts by weight of the hydrogel composition, wherein the additive comprises at least one selected from the group consisting of a cell, cell growth factor, buffer, preservative, isotonicity regulator, salt, antioxidant, osmotic pressure regulator, emulsifier, wetting agent, sweetener, flavoring agent, and anesthetic agent.

8. The hydrogel composition according to claim 7, wherein the cell comprises at least one selected from the group consisting of a stem cell, sensory cell, brain cell, germ cell, epithelial cell, and immune cell.

9. A hydrogel support for tissue regeneration, prepared using the hydrogel composition according to claim 1.
